(19) United States
(12) Patent Application Publication
(10) Pub. No.: US 2024/0177842 A1
(43) Pub. Date: May 30, 2024

(54) COMPUTATIONAL MODELS TO PREDICT FACILITY SCORES

(71) Applicant: MatrixCare, Inc., Bloomington, MN (US)

(72) Inventor: Vivek KUMAR, Eden Prairie, MN (US)

(21) Appl. No.: 18/059,781

(22) Filed: Nov. 29, 2022

(51) Int. Cl. G16H 40/20; G06Q 10/0639

(52) U.S. Cl. CPC G16H 40/20; G06Q 10/06393

(57) ABSTRACT

Techniques for improved predictive modeling are provided. Electronic health record (EHR) data for a plurality of patients associated with a first healthcare facility is collected. One or more predicted scores for the first healthcare facility are generated, using a prediction model, based on the EHR data, where the predicted scores are indicative of quality of the first healthcare facility. A set of features, from the EHR data, is ranked based on their salience to the one or more predicted scores. The ranked set of features is output via a graphical user interface (GUI).

### INTRODUCTION

Embodiments of the present disclosure relate to computer modeling. More specifically, embodiments of the present disclosure relate to predicting facility operation scores using computer modeling.

In many conventional healthcare settings, such as residential care facilities (e.g., nursing homes), hospitals, in-patient or out-patient clinics, and the like, facility operations scores are generated, provided, and/or tracked to monitor the efficacy, efficiency, and overall quality of care and operations. For example, for a long-term residential care facility, surveys of residents and/or staff may be used to gather information (e.g., asking each user to rate their experience out of five). These survey responses can be aggregated to generate overall ratings or scores for the facility (e.g., indicating the quality of care provided, or how well the facility is operating). Often, the specific algorithms used to generate such ratings are entirely opaque, preventing administrators from engaging with the ratings and data, and understanding how to improve. Further, the underlying metrics are generally inherently subjective and fickle, and require substantial time to collect (e.g., waiting for survey responses), preventing adequate evaluation.

Improved systems and techniques to automatically evaluate facility operations are needed.

### SUMMARY

According to one embodiment presented in this disclosure, a method is provided. The method includes: collecting electronic health record (EHR) data for a plurality of patients associated with a first healthcare facility; generating one or more predicted scores for the first healthcare facility, using a prediction model, based on the EHR data, wherein the predicted scores are indicative of quality of the first healthcare facility; ranking a set of features, from the EHR data, based on their salience to the one or more predicted scores; and outputting the ranked set of features via a graphical user interface (GUI).

According to one embodiment presented in this disclosure, a method is provided. The method includes: collecting a plurality of electronic health record (EHR) data for a plurality of healthcare facilities; determining a plurality of scores for the plurality of healthcare facilities. training a prediction model to generate predicted scores based on the plurality of EHR data and the plurality of scores, the predicted scores indicative of quality of the healthcare facility; and deploying the prediction model to generate predicted scores for healthcare facilities.

The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 depicts an example workflow for generating prediction models based on facility data, according to some embodiments.

FIG. 2 depicts an example workflow for evaluating facility data and generating predicted scores using prediction models, according to some embodiments.

FIG. 3 depicts an example workflow for dynamically generating updated predictions using newly received data, according to some embodiments.

FIG. 4 depicts an example workflow for identifying salient or impactful data affecting score predictions based on local evaluation, according to some embodiments.

FIG. 5 depicts an example workflow for identifying salient or impactful data affecting score predictions based on global evaluation, according to some embodiments.

FIG. 6 depicts a graphical user interface for monitoring and engaging with predicted operational scores, according to some embodiments.

FIG. 7 is a flow diagram depicting an example method for generating predictive models for operational scoring, according to some embodiments.

FIG. 8 is a flow diagram depicting an example method for generating predicted scores using predictive models, according to some embodiments.

FIG. 9 is a flow diagram depicting an example method for ranking impactful features using local evaluations, according to some embodiments.

FIG. 10 is a flow diagram depicting an example method for ranking impactful features using global evaluations, according to some embodiments.

FIG. 11 is a flow diagram depicting an example method for generating updated predictions for newly received data, according to some embodiments.

FIG. 12 is a flow diagram depicting an example method for refining predictive models over time, according to some embodiments.

FIG. 13 is a flow diagram depicting an example method for scoring and ranking features using predictive models, according to some embodiments.

FIG. 14 is a flow diagram depicting an example method for training predictive models to predict operational scores, according to some embodiments.

FIG. 15 depicts an example computing device configured to perform various aspects of the present disclosure, according to some embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for improved predictive models to evaluate facility data and predict operational efficacies and qualities.

In some embodiments, operational data from multiple facilities can be aggregated and used to build predictive models that enable more accurate and reliable evaluation of facility quality and operational efficacies and efficiencies. In some embodiments, healthcare facilities (e.g., residential care facilities) are used as example facilities where the presently disclosed techniques can be applied. Generally, however, aspects of the present disclosure are readily applicable to a wide variety of deployments. The particular data used to generate and/or use the models may vary depending on the particular implementation. For example, in a healthcare setting, the systems may collect and evaluate electronic health record (EHR) data from each facility.

In some embodiments, healthcare facilities can be rated or scored (e.g., on a zero to five star system) based on survey responses. Using techniques described herein, more granular or low-level data can be collected (e.g., from EHR data) to help predict and/or improve such ratings or scores. For example, in some embodiments, the system can identify variables (in the EHR or other data) that differ between low-rated facilities (also referred to as low quality, low performing, and/or low scored facilities) and high-rated facilities (also referred to as high performing, high quality, and/or high score facilities) in order to identify factors that might be causal or relevant to the overall scores. In some embodiments, the system can generate and/or train predictive models (e.g., machine learning models) using the assigned scores as the label/target output, and the low-level EHR data as the input. The model may thereby learn to predict facility scores based on specific facility data (e.g., to predict the quality of the facility based on its EHR). In some embodiments, a variety of techniques can then be used to discover or predict which input feature(s) are most salient or relevant for a given facility that desires to improve its score.

In embodiments, the particular data being evaluated may vary depending on the particular implementation. In some embodiments, the data from a given facility can be aggregated or evaluated to generate or extract features used for model creation and inferencing. For example, in one embodiment, the system can generate or extract information (e.g., from EHR data) indicating features such as the number or frequency of progress notes recorded (e.g., by a care provider) per resident or patient of the facility (e.g., the average, median, or total number of notes recorded in a defined window, such as per month), the number of times or frequency with which vitals are recorded for each patient or resident (e.g., the average, median, or total number of times the vitals, such as heartrate, blood pressure, and the like are collected over a duration, such as a month, for each resident), and the like.

As additional (non-limiting) examples of input data (or features generated or extracted from the input data) that may be used in various embodiments, the features may information relating to missed point-of-care (POC) documentation (e.g., the number or frequency of times when POC documentation is not recorded, or is not recorded within a defined timeline from when the care was provided), information relating to medication administration issues (e.g., the number or frequency of times when medications are administered late or not at all to one or more residents), information relating to payment or insurance claim rejections (e.g., the number or percentage of insurance claims that are rejected), information relating to hospitalizations (e.g., the re-hospitalization rate of residents in the facility), information relating to falls or other adverse events in the facility (e.g., the number or frequency of falls experienced by residents), information relating to infections in the facility (e.g., the infection rate), information relating to the quality of recorded notes (e.g., nursing notes), such as their average length, clarity, and/or depth, or any other information indicating or relating to the quality of the facility.

Such features may then be used to predict and/or evaluate the quality, efficiency, and/or efficacy of the facility, and may further be evaluated to identify which elements, if changed, would most impact the score or quality of the facility's operations. For example, the system may determine that the number of times vitals are recorded is in line with expectations or would not substantially affect the facility's score, but that increasing the number of progress notes made for each resident would likely have a significant positive effect on the quality of care/score of the facility.

In some embodiments, the generated predictive models can include dynamic or trained models (e.g., machine learning models such as regression-based models, neural networks, random forests, and the like), static or fixed models (e.g., algorithmic, rules-based, and the like), or hybrid models (e.g., a computational model that has both static and dynamic portions). Advantageously, by using such modeling, embodiments of the present disclosure are able to provide a variety of technical improvements over conventional solutions. For example, by automatically collecting and evaluating specific low-level data to generate new features (such as the number of progress notes per resident in the facility or the frequency of issues in medication administration) not reflected in conventional architectures, some embodiments of the present disclosure are able to provide more insightful and reliable predictions and recommendations. Similarly, by using objective data modeling (rather than subjective data such as survey responses), some embodiments of the present disclosure can enable more accurate and stable predictions.

Moreover, by automatically (and objectively) evaluating and ranking various features, some embodiments of the present disclosure are able to provide more targeted and specific updates, enabling efficient allocation of resources (including computational resources, such as computing time and storage, as well as physical resources, such as staff time and medical supplies). This reduces waste and improves the efficiency of the computing systems themselves, as well as the efficiency of the healthcare facilities. Additionally, using such targeted interventions, the healthcare outcomes or results for each individual resident or patient can be similarly improved. For example, hospitalization rates may be decreased, overall resident satisfaction or happiness may increase, and the like.

### Example Workflow for Generating Prediction Models Based on Facility Data

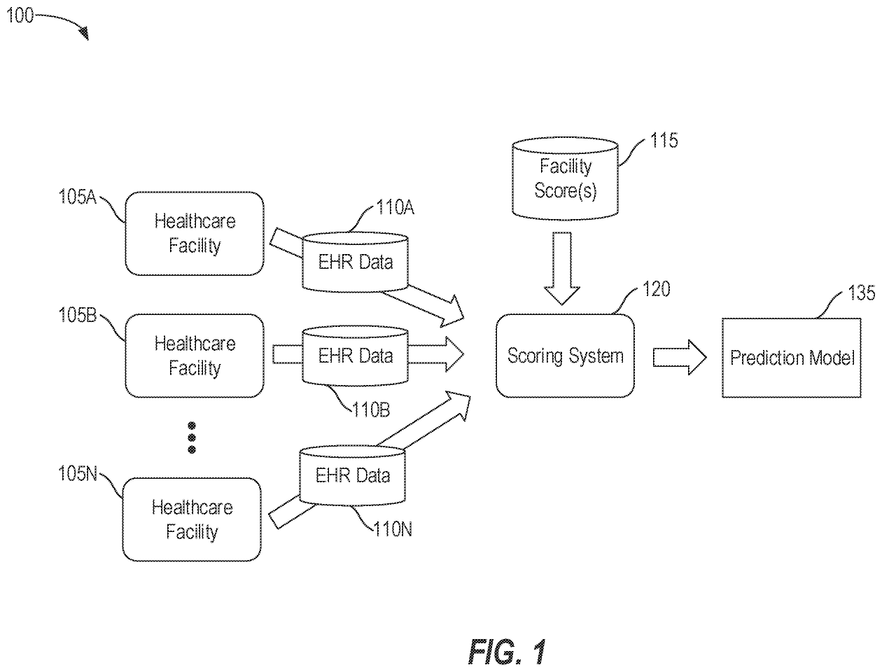

FIG. 1 depicts an example workflow 100 for generating prediction models based on facility data, according to some embodiments.

In the illustrated example, EHR data 110A-N from a variety of healthcare facilities 105A-N is collected and/or accessed by a scoring system 120. Though illustrated as a discrete system for conceptual clarity, in embodiments, the scoring system 120 may be integrated into one or more broader components or systems. For example, the scoring system 120 may be implemented as a component or application on a cloud system, on a system maintained or operated by one or more healthcare facilities 105, and the like. Further, the operations of the scoring system 120 may be implemented using hardware, software, or a combination of hardware and software.

In some embodiments, the healthcare facilities 105A-N (collectively, healthcare facilities 105) correspond to long-term residential care facilities (e.g., nursing homes or senior living facilities). In some embodiments, as discussed above, the healthcare facilities 105 may additionally or alternatively include or correspond to other healthcare facilities (e.g., clinics, hospitals, and the like) or to non-healthcare facilities or data sources. Generally, embodiments of the present disclosure are readily applicable to any environment where data from a deployment or entity can be collected, transformed, and/or evaluated to determine or predict quality or efficacy of the deployment or entity.

Although three discrete healthcare facilities 105 are depicted in the illustrated example, in embodiments the EHR data 110 (or other data) may be accessed or collected for any number of healthcare facilities 105 (or other facilities or data sources). Additionally, although the illustrated example depicts the EHR data 110 being received directly from the healthcare facilities 105, in some aspects, some or all of the data may be maintained and/or accessed from other sources. For example, EHR data 110A for the healthcare facility 105A may be stored or maintained via one or more external storage solutions (e.g., in the cloud), and the scoring system 120 may access the EHR data 110A from such sources, rather than directly from the healthcare facility 105A. As used herein, accessing data may generally include receiving, requesting, retrieving, or otherwise gaining access to the data.

In an embodiment, the EHR data 110 comprises or corresponds to various records relating to health information for various patients or residents. For example, the EHR data 110A may include such information for residents or patients of healthcare facility 105A. Generally, the particular contents of the EHR data 110 may vary depending on the particular implementation. In some embodiments, the EHR data 110 may include, for one or more patients or residents, information such as their demographics, medications, test results, allergies, medical history, vital signs collected or recorded at one or more times, progress notes recorded (e.g., by a care provider) at one or more times, and the like.

In some embodiments, in addition to or instead of including patient-specific data, the EHR data 110 may include (or be used to generate) aggregated data or features for the healthcare facility 105. For example, the scoring system 120 (or another system) may determine aggregated attributes such as the average number of progress notes recorded per resident over a defined window or duration of time (e.g., the last month), the average demographics (e.g., average age) of the residents of the facility, the aggregated medication usage or administration issues of the residents of the facility, and the like. In some embodiments, the aggregated features relate to operational or mutable elements (such as the average time spent with each resident per day) rather than patient-specific elements (such as the average age of the residents).

In the illustrated workflow 100, the scoring system 120 further receives or accesses a set of facility scores 115. The facility scores 115 generally comprise or correspond to one or more measures that quantify the healthcare facilities 105 based on their operations. For example, the facility scores 115 may quantify the quality, efficiency, efficacy, or other aspect of the healthcare facilities 105. In some embodiments, a higher facility score 115 corresponds to a higher quality facility. For example, a healthcare facility 105 with a facility score 115 of 4.8 may be considered higher quality, more efficient, more effective, higher performing, or otherwise better than a healthcare facility 105 with a facility score 115 of 1.4. In some embodiments, as discussed above, the facility scores 115 may be generated or created based on surveys or other data from residents, patients, and/or staff at each healthcare facility 105.

For example, the scoring system 120 (or another system) may provide a survey to the residents/staff, asking each to respond to questions using numerical scores, written responses, or other input to quantify or explain how well their healthcare facility 105 is operating. Based on the responses, the scoring system 120 (or another system) can thereby generate facility scores 115 quantifying the quality of each healthcare facility 105 across one or more dimensions. For example, for a given healthcare facility 105, the facility scores 115 may indicate a score or measure of the quality of resident outcomes, a score or measure of the comfort experienced by residents, a score or measure of the workload of staff, and the like. In at least one embodiment, the facility scores 115 may be referred to as “real scores” or “actual scores” to differentiate them from generated/predicted scores.

As depicted, the scoring system 120 can use the accessed facility scores 115 and EHR data 110 to train, refine, or otherwise generate or create one or more prediction models 135. As discussed above, the prediction models 135 can generally include static model component(s) (e.g., rules-based or algorithmic components), dynamic model component(s) (e.g., trained or learned components), or a combination of both (e.g., hybrid models). Generally, the prediction models 135 are configured to generate or output a predicted facility score based on input EHR data.

For example, during training, the scoring system 120 may process EHR data 110 from a given healthcare facility 105 as input to the prediction model 135 in order to generate one or more output predicted scores. Such generated score(s) can then be compared against the corresponding facility score(s) 115 for the healthcare facility 105, and one or more components of the prediction model 135 (e.g., weights, biases, or other parameters) can be updated or refined based on this difference. In this way, the prediction model 135 is iteratively refined using EHR data 110 and facility scores 115 to generate more accurate predictions.

In some embodiments, some or all of the EHR data 110 may be provided directly to the prediction model 135. In some embodiments, some or all of the EHR data 110 may be preprocessed prior to being used as input to the model. For example, as discussed above, the scoring system 120 may determine aggregated features for the healthcare facility 105, such as the average number of times vitals are recorded for each resident (or how frequently vitals are recorded, on average). This process of generating or determining aggregate (e.g., facility-wide) features may generally be referred to as feature extraction or generation in some embodiments.

In some embodiments, the scoring system 120 generates, trains, or refines the prediction model 135 during an initial training phase. Once trained, the scoring system 120 may deploy the prediction model for runtime use. For example, the prediction model 135 may be used to predict facility scores based on EHR data for a given facility, and/or identify features of the EHR data that, if changed, would have an impact on the scores (e.g., that would raise the score above a threshold amount). In this way, embodiments of the present disclosure enable significant improvements over conventional systems.

### Example Workflow for Evaluating Facility Data and Generating Predicted Scores Using Prediction Models

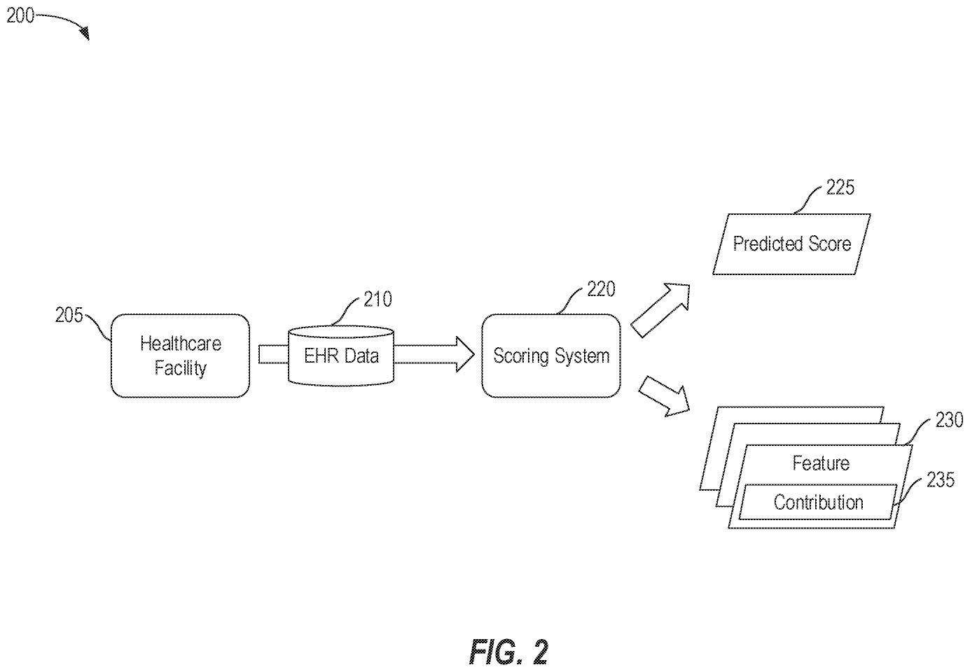

FIG. 2 depicts an example workflow 200 for evaluating facility data and generating predicted scores using prediction models, according to some embodiments.

In the illustrated example, a scoring system 220 accesses EHR data 210 from a healthcare facility 205 to generate predicted score(s) 225 and/or impactful feature(s) 230. Though illustrated as a discrete system for conceptual clarity, in embodiments, the scoring system 220 may be integrated into one or more broader components or systems. For example, the scoring system 220 may be implemented as a component or application on a cloud system, on a system maintained or operated by one or more healthcare facilities 205, and the like. Further, the operations of the scoring system 220 may be implemented using hardware, software, or a combination of hardware and software.

In some embodiments, the scoring system 220 may correspond to the scoring system 120 of FIG. 1. That is, a single scoring system may be used to generate the prediction models and to use the models during runtime. In other embodiments, the scoring system 220 may differ from the scoring system 120. That is, a first system may train or generate the models, and the models may be deployed to a second system for runtime use.

In some embodiments, the healthcare facility 205 corresponds to a long-term residential care facility (e.g., a nursing home or senior living center). In some embodiments, as discussed above, the healthcare facility 205 may additionally or alternatively include or correspond to other healthcare facilities (e.g., clinics, hospitals, and the like) or to non-healthcare facilities or data sources. In at least one embodiment, the healthcare facility 205 has the same type or overall architecture as the facilities used to generate the prediction model. That is, the healthcare facility 205 may in some way match or correspond to the healthcare facilities 105 of FIG. 1. For example, if the healthcare facilities 105 are long-term residential care centers, the healthcare facility 205 may similarly be a long-term residential care facility. In some embodiments, the healthcare facility 205 may itself be a healthcare facility from which data was used to train or generate the prediction model. In other embodiments, the healthcare facility 205 may be a separate facility that uses the deployed model, but which was not used to train or generate the model.

Although one discrete healthcare facility 205 is depicted in the illustrated example, in embodiments the scoring system 220 may access or collect EHR data 210 for any number of healthcare facilities 205 (or other facilities or data sources). Additionally, although the illustrated example depicts the EHR data 210 being received directly from the healthcare facility 205, in some aspects, some or all of the data may be maintained and/or accessed from other sources. For example, EHR data 210 for the healthcare facility 205 may be stored or maintained via one or more external storage solutions (e.g., in the cloud), and the scoring system 220 may access the EHR data 210 from such sources, rather than directly from the healthcare facility 205.

In an embodiment, as discussed above, the EHR data 210 comprises or corresponds to various records relating to health information for various patients or residents. For example, the EHR data 210 may include such information for residents or patients of healthcare facility 205. Generally, the particular contents of the EHR data 210 may vary depending on the particular implementation. In some embodiments, the EHR data 210 may include, for one or more patients or residents, information such as their demographics, medications, test results, allergies, medical history, vital signs collected or recorded at one or more times, progress notes recorded (e.g., by a care provider) at one or more times, and the like. In an embodiment, the EHR data 210 generally comprises data for the same variables or information used to train or generate the prediction models. For example, if the model was trained using information relating to the frequency with which vitals were recorded per patient, the EHR data 210 may include such information as well.

In some embodiments, in addition to or instead of including patient-specific data, the EHR data 210 may include (or be used to generate) aggregated data or features for the healthcare facility 205, as discussed above. For example, the scoring system 220 (or another system) may determine aggregated attributes or features such as the average number of progress notes recorded per resident over a defined window of time (e.g., the last month), the average demographics (e.g., average age) of the residents of the facility, the aggregated medication usage and/or issues with medication administration of the residents of the facility, and the like.

In some embodiments, the scoring system 220 corresponds to a central system or entity that manages multiple healthcare facilities 205. For example, a central management entity may use the scoring system 220 to evaluate the EHR data 210 from multiple such facilities, allowing a more global approach to resource use and load balancing.

In some embodiments, the scoring system 220 accesses the EHR data 210 continuously (e.g., as it is generated or created). In some embodiments, the scoring system 220 can access the EHR data 210 periodically (e.g., weekly), or upon the occurrence of some defined event(s) (e.g., when a user, such as an administrator or manager, requests that the scoring system 220 do so). This may reduce the computational burden on the scoring system 220 and/or on the systems that maintain the EHR data 210, and may further reduce network congestion caused by accessing the data. In at least one aspect, the scoring system 220 may access the EHR data 210 during specified times (e.g., overnight, or other times when network traffic or other burden on the computing systems is low) to reduce the potential for interference with other processes.

In the illustrated example, the scoring system 220 processes the EHR data 210 (or generated or transformed features from the EHR data 210) using one or more prediction models (e.g., prediction model 135 of FIG. 1) to generate one or more predicted scores 225 for the healthcare facility 205 and one or more salient or impactful features 230 for the healthcare facility 205. As discussed above, the predicted score 225 generally quantifies or indicates the (predicted) quality of the healthcare facility 205, where higher scores are generally indicative of higher quality.

In this way, using generated computational models, the scoring system 220 can allow rapid and dynamic prediction of facility scores. In contrast, conventional solutions (e.g., survey-based approaches) are inherently limited and time-consuming (in addition to being inherently subjective). That is, conventional approaches to determine the facility score can often take substantial time (e.g., months or even years) to complete, such that the facility itself has often changed significantly during the score generation process. Conventional scores are therefore often outdated or irrelevant. In contrast, the scoring system 220 can generate new scores rapidly and automatically (e.g., daily, weekly, or monthly), enabling far more rapid quantification of facility quality, identification of potential concerns, and overall improved response.

For example, managers of the healthcare facility 205 may use the scoring system 220 to generate updated predicted scores 225 at the end of each shift or week, allowing them to efficiently monitor the progress or quality of the facility at the instant moment and over time. This enables deeper insights, as well as a more accurate and complete understanding of the efficiency and efficacy of the current configurations (e.g., staff assignments, resource usage, and the like).

In the illustrated example, the scoring system 220 further identifies features 230. Generally, each feature 230 corresponds to some aspect of the healthcare facility 205 (e.g., reflected in the EHR data 210) that has some impact (large or small) on the predicted score 225. In some embodiments, the features 230 correspond to those aggregated features (generated based on EHR data 210) that are used as input to the model. For example, a first feature 230 may correspond to the median frequency with which vitals are recorded for each resident, while a second feature 230 may correspond to the average number of progress notes recorded, per resident, per shift, and a third feature 230 may correspond to the resident-to-staff ratio.

As illustrated, each feature 230 further includes a corresponding measure of its contribution 235 to the predicted score(s) 225. The contributions 235 generally indicate how impactful the corresponding feature 230 is on the predicted score 225. For example, a feature 230 having a high contribution 235 may be significant (e.g., where small changes to the feature 230 result in substantially different predicted scores 225), while a feature 230 having a lower contribution 235 may be insignificant (e.g., where changing the feature 230 has little or no effect on the predicted score 225).

In embodiments, the scoring system 220 may generate the contributions 235 using a wide variety of suitable techniques. For example, in some embodiments, the scoring system 220 uses feature selection operations or techniques (during training or runtime) to identify the feature(s) that are most impactful on the model outputs, and generates the contributions 235 accordingly.

In some embodiments, to determine the contributions 235, the scoring system 220 can generate multiple predicted scores 225 for the healthcare facility 205 based on perturbations to the EHR data 210 (e.g., a first score for the unmodified data, a second score for the data with one or more features modified randomly, and the like). By evaluating the difference(s) between the perturbed and original data and the resulting differences between the predicted scores 225, the scoring system 220 can infer or determine which feature, or combinations of features, are most impactful.

In some embodiments, to determine the contributions 235, the scoring system 220 can compare the EHR data from one or more high-scoring facilities with that of one or more low-scoring facilities. In this way, the scoring system 220 may identify which feature(s) 230 tend to differ significantly between the two sets of facilities, and thereby infer or determine which feature(s) 230 are most impactful or most likely to result in higher predicted scores 225.

In some embodiments, the scoring system 220 outputs an indication of all the features 230, each with a corresponding contribution 235. In some embodiments, the scoring system 220 can rank/sort and/or filter the features 230 based on the contributions 235. For example, the scoring system 220 may indicate the five highest-scored features 230 (e.g., those having the highest contributions 235), allowing users to efficiently identify which action(s) should be taken or which feature(s) 230 are most relevant for the specific healthcare facility 205 at the specific time.

That is, the scoring system 220 can enable the user(s) to immediately identify not only the predicted facility quality (via the predicted score 225), but also to identify which feature(s) are the cause of the score (e.g., which aspects or configurations should be changed, how they should be changed, which aspects should be maintained, and the like). This can allow rapid remediation of any concerns, significantly improving resident outcomes and resource usage, and further reducing waste.

Example Workflow for Dynamically Generating Updated Predictions using Newly Received Data

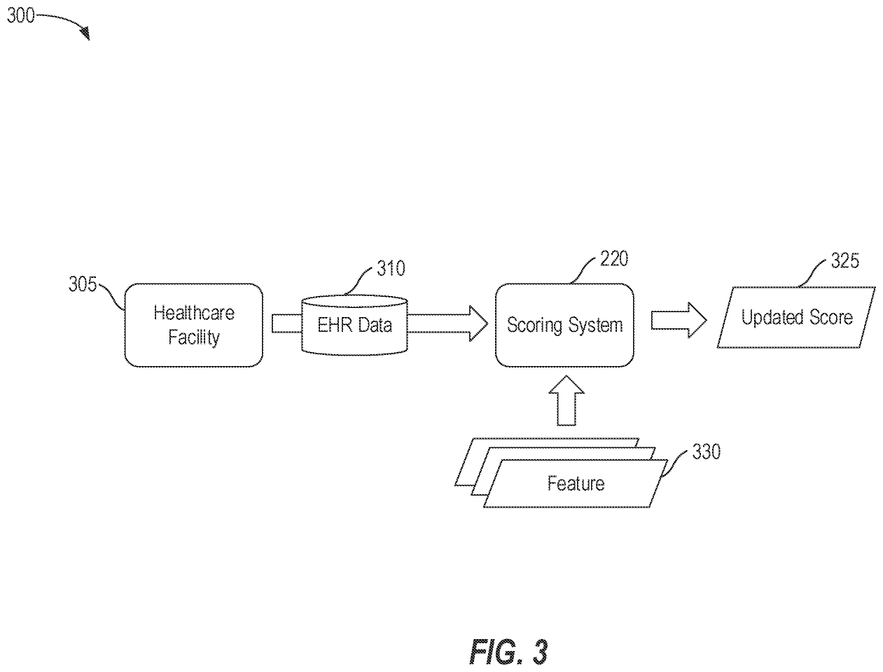

FIG. 3 depicts an example workflow 300 for dynamically generating updated predictions using newly received data, according to some embodiments.

In the illustrated example, a scoring system 220 accesses EHR data 310 for a healthcare facility 305, along with updated features 330, to generate predicted score(s) 325. In some embodiments, the healthcare facility 305 corresponds to a long-term residential care facility (e.g., a nursing home or senior living center). In some embodiments, as discussed above, the healthcare facility 305 may additionally or alternatively include or correspond to other healthcare facilities (e.g., clinics, hospitals, and the like) or to non-healthcare facilities or data sources. In at least one embodiment, the healthcare facility 305 has the same type or overall architecture as the facilities used to generate the prediction model. That is, the healthcare facility 305 may in some way match or correspond to the healthcare facilities 105 of FIG. 1. For example, if the healthcare facilities 105 are long-term residential care centers, the healthcare facility 305 may similarly be a long-term residential care facility. In some embodiments, the healthcare facility 305 may itself be a healthcare facility from which data was used to train or generate the prediction model. In other embodiments, the healthcare facility 305 may be a separate facility that uses the deployed model, but which was not used to train or generate the model.

In at least one embodiment, the healthcare facility 305 corresponds to the healthcare facility 205 of FIG. 2. For example, as discussed above with reference to FIG. 2, EHR data from the healthcare facility may be used to generate predicted score(s) and/or impactful feature(s). In the illustrated example, updated features 330 (e.g., new values for one or more features) may be received and evaluated to generate updated scores 325. For example, as discussed below in more detail, the updated features 330 may correspond to user-defined features or updates to the EHR 310, such as aspirational attributes for the healthcare facility 305, or may correspond to new data (e.g., collected since the last time the predicted score was generated).

Although one discrete healthcare facility 305 is depicted in the illustrated example, in embodiments the scoring system 220 may access or collect EHR data 310 for any number of healthcare facilities 305 (or other facilities or data sources). Additionally, although the illustrated example depicts the EHR data 310 being received directly from the healthcare facility 305, in some aspects, some or all of the data may be maintained and/or accessed from other sources. For example, EHR data 310 for the healthcare facility 305 may be stored or maintained via one or more external storage solutions (e.g., in the cloud), and the scoring system 220 may access the EHR data 310 from such sources, rather than directly from the healthcare facility 305.

In an embodiment, as discussed above, the EHR data 310 comprises or corresponds to various records relating to health information for various patients or residents. For example, the EHR data 310 may include such information for residents or patients of healthcare facility 305. Generally, the particular contents of the EHR data 310 may vary depending on the particular implementation. In some embodiments, the EHR data 310 may include, for one or more patients or residents, information such as their demographics, medications, test results, allergies, medical history, vital signs collected or recorded at one or more times, progress notes recorded (e.g., by a care provider) at one or more times, and the like. In an embodiment, the EHR data 310 generally comprises data for the same variables or information used to train or generate the prediction models. For example, if the model was trained using information relating to the frequency with which vitals were recorded per patient, the EHR data 310 may include such information as well.

In some embodiments, in addition to or instead of including patient-specific data, the EHR data 310 may include (or be used to generate) aggregated feature data for the healthcare facility 305, as discussed above. For example, the scoring system 220 (or another system) may determine aggregated attributes such as the average number of progress notes recorded per resident over a defined window of time (e.g., the last month), the average demographics (e.g., average age) of the residents of the facility, the aggregated medication usage and/or issues with medication administration of the residents of the facility, and the like.

In some embodiments, the scoring system 220 accesses the EHR data 310 continuously (e.g., as it is generated or created). In some embodiments, the scoring system 220 can access the EHR data 310 periodically (e.g., weekly), or upon the occurrence of some defined event(s) (e.g., when a user, such as an administrator or manager, requests that the scoring system 220 do so). This may reduce the computational burden on the scoring system 220 and/or on the systems that maintain the EHR data 310, and may further reduce network congestion caused by accessing the data. In at least one aspect, the scoring system 220 may access the EHR data 310 during specified times (e.g., overnight, or other times when network traffic or other burden on the computing systems is low) to reduce the potential for interference to other processes.

In the illustrated example, the scoring system 220 processes the EHR data 310 (or generated or transformed features from the EHR data 310), along with the updated features 330, using one or more prediction models (e.g., prediction model 135 of FIG. 1) to generate one or more updated scores 325 for the healthcare facility 305. As discussed above, the predicted score 325 generally quantifies or indicates the (predicted) quality of the healthcare facility 305, where higher scores are generally indicative of higher quality.

In some embodiments, as discussed above, the features 330 correspond to newly determined or generated data, and/or to new values for the data. For example, in response to determining that a given feature is impactful (e.g., because it has a high contribution, as discussed above with reference to FIG. 2), a user (e.g., an administrator) may define a potential or speculative updated value for the feature. For example, the user may indicate or request that an updated score 325 be generated to reflect the facility quality if the resident vitals were taken weekly, rather than monthly. The resulting updated scores 325 can be used to more efficiently drive configurations and allocations in the healthcare facility 305 (e.g., to identify which aspects should be changed, how much they should or can be changed, who many resources should be dedicated to the change, and the like). Alternatively, the features 330 may be used to indicate actual updates or changes, such as reflected in new data that was not available when the original score was generated.

In these ways, the scoring system 220 can provide dynamic and interactive updated scores 325, allowing users to efficiently answer “what-if” questions. That is, the user can review the current (predicted) score, suggest updated features 330 to see how the score changes, and immediately test these updates. This can significantly improve the facility operations and overall efficacy, allowing rapid remediation and reconfiguration of the overall system.

### Example Workflow for Identifying Salient or Impactful Data Affecting Score Predictions Based on Local Evaluation

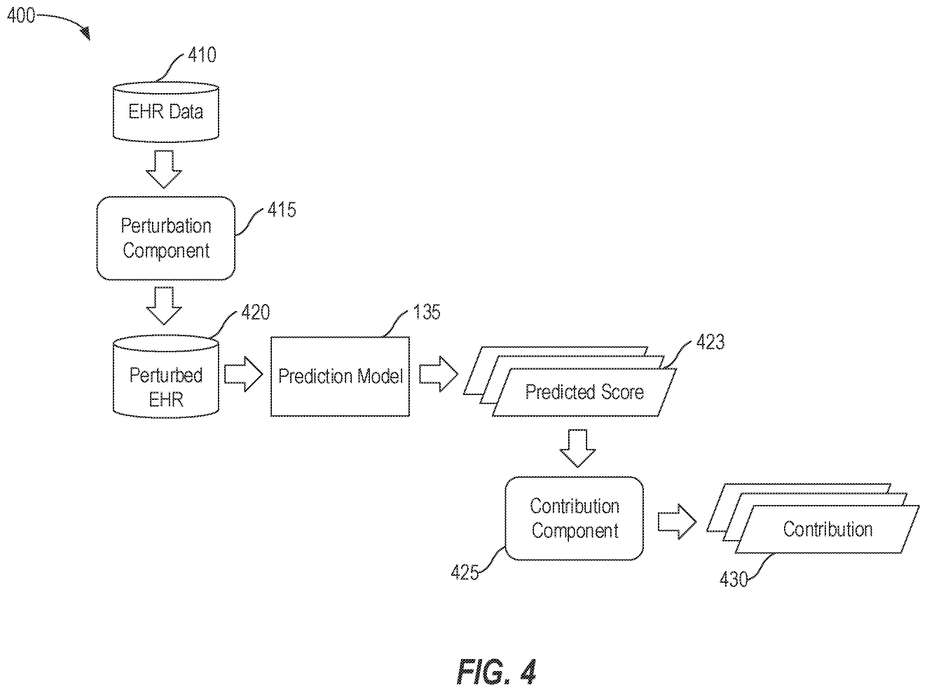

FIG. 4 depicts an example workflow 400 for identifying salient or impactful data affecting score predictions based on local evaluation, according to some embodiments. In some embodiments, the workflow 400 can be used to determine the contribution (e.g., contributions 235 of FIG. 2) of each feature on the overall predicted scores.

In the illustrated example, a perturbation component 415 and contribution component 425 perform various operations to generate contributions 430 based on EHR data 410. Although depicted as discrete components for conceptual clarity, in embodiments, the operations of the perturbation component 415 and contribution component 425 may be combined or distributed across any number of components. Further, the operations of the perturbation component 415 and contribution component 425 may be implemented using hardware, software, or a combination of hardware and software. In at least one embodiment, the perturbation component 415 and contribution component 425 are components of a scoring system, such as the scoring system 120 of FIG. 1 and/or the scoring system 220 of FIG. 2.

As illustrated, EHR data 410 for one or more facilities (e.g., healthcare facilities) is accessed by the perturbation component 415. As discussed above, the EHR data 410 may comprise or correspond to various records relating to health information for various patients or residents. For example, the EHR data 410 may include such information for residents or patients of a healthcare facility. Generally, the particular contents of the EHR data 410 may vary depending on the particular implementation. In some embodiments, the EHR data 410 may include, for one or more patients or residents, information such as their demographics, medications, test results, allergies, medical history, vital signs collected or recorded at one or more times, progress notes recorded (e.g., by a care provider) at one or more times, and the like. In an embodiment, the EHR data 410 generally comprises data for the same variables or information used to train or generate the prediction models. For example, if the model was trained using information relating to the frequency with which vitals were recorded per patient, the EHR data 410 may include such information as well.

In some embodiments, in addition to or instead of including patient-specific data, the EHR data 410 may include (or be used to generate) aggregated feature data for the healthcare facility, as discussed above. These aggregated features can then be used as the model input.

In the illustrated example, the perturbation component 415 can modify or transform the EHR data 410 (e.g., the aggregated features) to generate one or more sets of perturbed EHR 420. In some embodiments, these perturbations correspond to modified values for one or more features in the EHR data 410. For example, the perturbation component 415 may use one or more techniques (including random or pseudo-random techniques) to modify one or more values in the original set of EHR data 410 to generate a set of perturbed EHR 420. In at least one aspect, the perturbations are defined based on the distribution of values reflected in the EHR data for a number of facilities. For example, the perturbation component 415 may perturb the values probabilistically, ensuring that they fall within reasonable or realistic bounds.

In some embodiments, the perturbation component 415 generates multiple sets of perturbed EHR 420, where each set may have perturbed values for one or more individual features. That is, each set of perturbed EHR 420 may generally have the same values as the EHR data 410 for one or more features, but may have differing values for one or more other features. As illustrated, the perturbed EHR 420 can then be processed using the prediction model 135 to generate a set of predicted scores 423 (e.g., one for each set of perturbed EHR 420).

In this way, the scoring system can generate not only a predicted score for the actual EHR data 410, but also a respective score for each set of perturbed EHR 420. As illustrated, the contribution component 425 can thereafter access and evaluate the predicted scores 423 in order to generate contributions 430.

In one embodiment, based on the difference(s) between the predicted scores 423 and the underlying values of each set of perturbed EHR 420, the contribution component 425 may identify the contribution or impact of each perturbed feature. For example, the contribution component 425 may identify one or more sets of perturbed EHR 420 having the highest predicted score(s) 423, and compare the features of the perturbed EHR 420 to the original EHR data 410. The combination of determined differences may be used to define contributions 430 (e.g., based on the magnitude of the differences for each feature and/or the magnitude of the difference in predicted scores).

That is, the contribution 430 of a given feature may depend on both the magnitude of the applied perturbation to the feature (e.g., how much it differs from the original EHR data 410) as well as the magnitude of the score difference (e.g., how much the predicted score 423 for the perturbed EHR 420 differs from the predicted score for the real EHR data 410). In this way, by evaluating multiple (perturbed) alternatives or modifications, the system may automatically identify the contributions 430 of each feature, and/or quantify and indicate which feature(s) should be changed in improve the predicted score, as well as by what amount.

### Example Workflow for Identifying Salient or Impactful Data Affecting Score Predictions Based on Global Evaluation

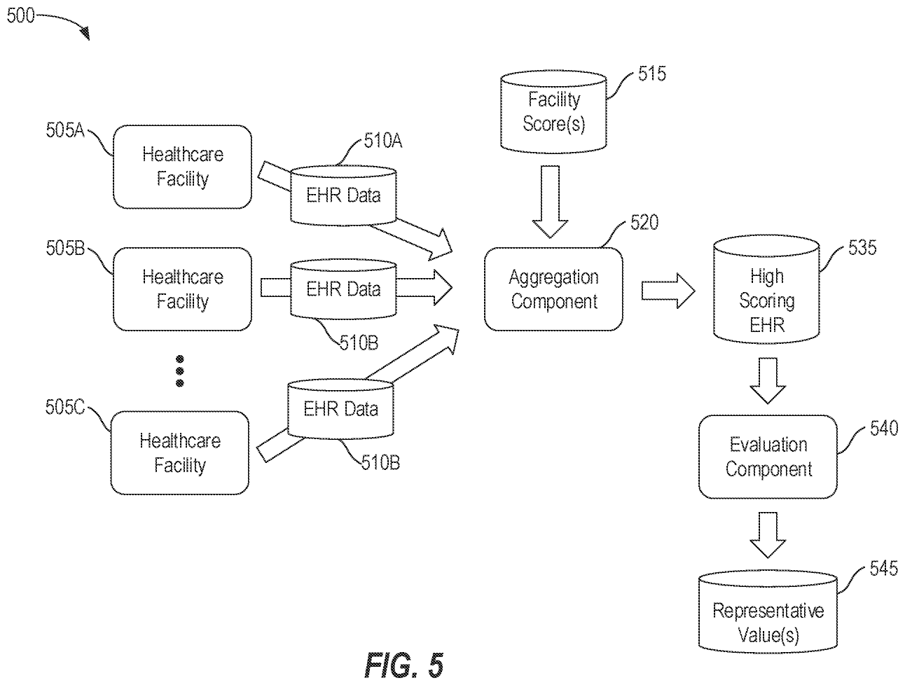

FIG. 5 depicts an example workflow 500 for identifying salient or impactful data affecting score predictions based on global evaluation, according to some embodiments. In some embodiments, the workflow 500 can be used to determine the contribution (e.g., contributions 235 of FIG. 2) of each feature on the overall predicted scores.

In the illustrated example, an aggregation component 520 and evaluation component 540 perform various operations to generate representative values 545 based on EHR data 510. Although depicted as discrete components for conceptual clarity, in embodiments, the operations of the aggregation component 520 and evaluation component 540 may be combined or distributed across any number of components. Further, the operations of the aggregation component 520 and evaluation component 540 may be implemented using hardware, software, or a combination of hardware and software. In at least one embodiment, the aggregation component 520 and evaluation component 540 are components of a scoring system, such as the scoring system 120 of FIG. 1 and/or scoring system 220 of FIG. 2.

In the illustrated example, EHR data 510A-N from a variety of healthcare facilities 505A-N is collected and/or accessed by the aggregation component 520. In some embodiments, as discussed above, the healthcare facilities 505A-N (collectively, healthcare facilities 505) correspond to long-term residential care facilities (e.g., nursing homes or senior living facilities). In some embodiments, as discussed above, the healthcare facilities 505 may additionally or alternatively include or correspond to other healthcare facilities (e.g., clinics, hospitals, and the like) or to non-healthcare facilities or data sources. Generally, embodiments of the present disclosure are readily applicable to any environment where data from a deployment or entity can be collected, transformed, and/or evaluated to determine or predict quality of efficacy of the deployment or entity.

Although three discrete healthcare facilities 505 are depicted in the illustrated example, in embodiments the EHR data 510 (or other data) may be accessed or collected for any number of healthcare facilities 505 (or other facilities or data sources). Additionally, although the illustrated example depicts the EHR data 510 being received directly from the healthcare facilities 505, in some aspects, some or all of the data may be maintained and/or accessed from other sources. For example, EHR data 510 for each healthcare facility 505 may be stored or maintained via one or more external storage solutions (e.g., in the cloud), and the aggregation component 520 may access the EHR data 510 from such sources, rather than directly from the healthcare facility 505.

In an embodiment, as discussed above, the EHR data 510 comprises or corresponds to various records relating to health information for various patients or residents. For example, the EHR data 510 may include such information for residents or patients of healthcare facility 505. Generally, the particular contents of the EHR data 510 may vary depending on the particular implementation. In some embodiments, the EHR data 510 may include, for one or more patients or residents, information such as their demographics, medications, test results, allergies, medical history, vital signs collected or recorded at one or more times, progress notes recorded (e.g., by a care provider) at one or more times, and the like. In an embodiment, the EHR data 510 generally comprises data for the same variables or information used to train or generate the prediction models. For example, if the model was trained using information relating to the frequency with which vitals were recorded per patient, the EHR data 510 may include such information as well.

In some embodiments, in addition to or instead of including patient-specific data, the EHR data 510 may include (or be used to generate) aggregated feature data for the healthcare facility 505. For example, the aggregation component 520 (or another system) may determine aggregated attributes such as the average number of progress notes recorded per resident over a defined window of time (e.g., the last month), the average demographics (e.g., average age) of the residents of the facility, the aggregated medication usage of the residents and/or medication issues of the facility, and the like. These aggregated features may then be used as model input.

In the illustrated workflow 500, the aggregation component 520 further receives or accesses a set of facility scores 515. As discussed above, the facility scores 515 generally comprise or correspond to one or more measures that quantify the healthcare facilities 505 based on their operations. For example, the facility scores 515 may quantify the quality, efficiency, efficacy, or other aspect of the healthcare facilities 505. In some embodiments, a higher facility score 515 corresponds to a higher quality facility. In some embodiments, as discussed above, the facility scores 515 may be generated or created based on surveys or other data from residents, patients, and/or staff at each healthcare facility 505.

In the illustrated embodiment, the aggregation component 520 evaluates the EHR data 510 and facility scores 515 to generate a set of high scoring EHR 535. Generally, the high scoring EHR 535 corresponds to the EHR data 535 from healthcare facilities 505 having a facility score 515 that satisfies one or more criteria, such as a threshold minimum value. That is, the aggregation component 520 may use the facility scores 515 to identify one or more healthcare facilities 505 that satisfy the criteria (e.g., that are sufficiently high quality), and the corresponding EHR data 510 from each high-quality healthcare facility 505 can be used to form the high scoring EHR 535.

As illustrated, the evaluation component 540 can then evaluate the high scoring EHR 535 to generate representative value(s) 545. In an embodiment, the representative values 545 indicate the aggregate or representative features of the high-scoring healthcare facilities 505. For example, the evaluation component 540 may determine aggregate values for each feature, such as the average value, median value, variance, or other information. In this way, the representative values 545 may be used to represent or indicate the feature values indicative of high facility quality.

As discussed above and in more detail below, when a user wishes to identify the contribution(s) of any features for a given facility (e.g., to identify which attributes are the cause of a low predicted quality score), the representative values 545 can be used to identify which feature value(s) (reflected in the EHR data for the specific facility) differ from the representative value(s) 545 of high-scoring facilities. In some embodiments, in addition to or instead of comparing the representative values to the given facility, the scoring system can similarly determine representative values for low scoring facilities in order to identify features that tend to differ substantially between high and low scoring facilities.

These features (determined based on the specific given facility, or based on low scoring facilities in general) may then be indicated or provided as the impactful or salient features and/or features having a high contribution or impact. This allows users to readily identify which feature(s) are causing a low score, as well as how they should be changed to better align with the high-scoring facilities (as indicated by the representative values 545).

Example Graphical User Interface for Monitoring and Engaging with Predicted Operational Scores

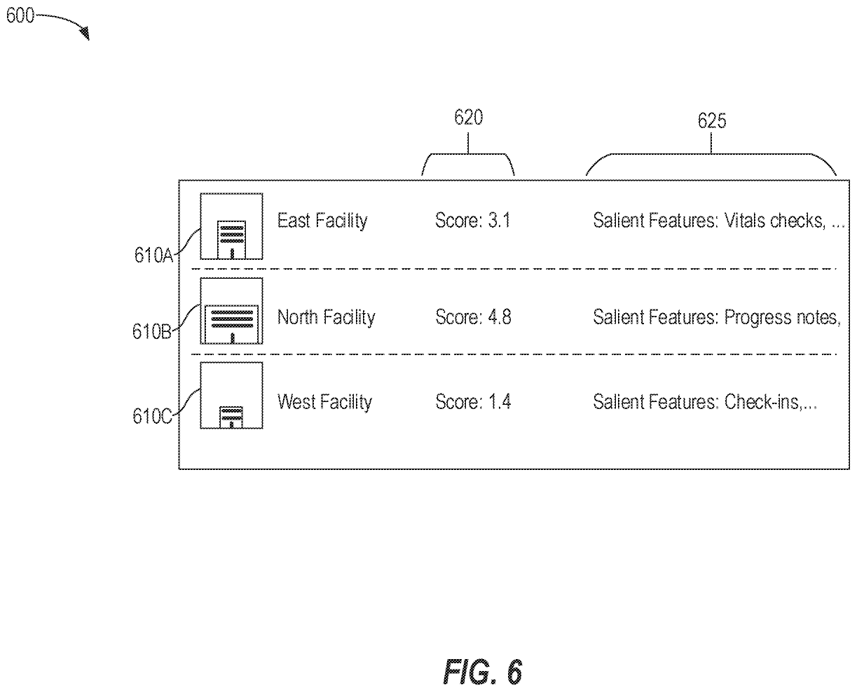

FIG. 6 depicts a graphical user interface (GUI) 600 for monitoring and engaging with predicted operational scores, according to some embodiments. In some embodiments, the GUI 600 is output or provided by a scoring system, such as the scoring system 120 of FIG. 1 and/or the scoring system 220 of FIGS. 2-3. In other embodiments, the GUI 600 is output by other systems (e.g., user terminals) based on data provided by or accessed from a scoring system.

In the illustrated example, the GUI 600 includes three portions 610A-C, one for each of three healthcare facilities. Although three such portions 610 are depicted, in embodiments, the GUI 600 may include similar portions 610 for any number of healthcare facilities. In at least one embodiment, the healthcare facilities correspond to those under the management or control of a single entity or manager. Generally, the individual facilities may correspond to discrete facilities (e.g., in different geographic areas), or different parts of the same facility (e.g., different buildings or wings in the same location).

In the illustrated GUI 600, each portion 610 includes identifying information for the corresponding facility, such as an image of the facility, a name of the facility, and the like. Further, as indicated by section 620, each portion 610 includes a facility score for the corresponding facility. Specifically, the “East Facility” in portion 610A has a facility score of 3.1, the “North Facility” in portion 610B has a facility score of 4.8, and the “West Facility” in 610C has a score of 1.4. In some embodiments, the indicated scores may be actual scores, such as generated using surveys, as discussed above. In some embodiments, the indicated scores may be predicted scores, such as generated using a prediction model, as discussed above.

In some embodiments, the GUI 600 may enable users to dynamically sort and/or filter the facilities based at least in part on their scores. For example, the GUI 600 may display the lowest-scoring facilities in a more prominent position (e.g., near the top) to allow for efficient identification of potentially problematic facilities that may need attention (while moving high-scoring facilities that do not need such attention to less prominent locations on the GUI 600, or removing them entirely), or may display the highest-scoring facilities in the more prominent position to allow for efficient identification of the highest-quality facilities (while moving low-scoring facilities that are not reflective of high quality to less prominent locations on the GUI 600, or removing them entirely).

In the illustrated example, as indicated by section 625, the GUI 600 further includes, for each portion 610, a corresponding set of salient features for each facility. As discussed above, salient features (also referred to as impactful features in some embodiments) are generally those that either have a significant impact on the predicted scores (e.g., have a high contribution) and/or that would be most impactful if they were changed. For example, the salient features may indicate attributes that, if they were changed, would have the most-significant positive effect on the score (e.g., to further increase the score), and/or those that are most responsible for the current score (e.g., those causing the current score).

In some embodiments, the salient features are determined based on determined contributions for each feature, such as determined using the workflow 400 of FIG. 4 and/or the workflow 500 of FIG. 5. In at least one embodiment, the salient features may differ depending on whether the facility is a high-scoring facility or a low-scoring facility (e.g., determined based on one or more thresholds). For example, for a high scoring facility (e.g., North Facility, depicted in portion 610B), such as those scored above a defined value, the salient features may be those that are most responsible for the high score. That is, though changing such features may be unlikely to substantially increase the score, they may nevertheless be useful to review in order to determine how to reconfigure lower-scoring facilities. As another example, for a low scoring facility (e.g., West Facility, depicted in portion 610C), such as those scored below a defined value, the salient features may be those that would be most impactful if changed. That is, the salient features may be those that, if changed, would have the largest impact on increasing the score.

In an aspect, the user may interact with the GUI 600 in a number of ways, such as by selecting one or more of the facilities (e.g., by clicking or tapping on them) in order to review more detail. For example, in response to receiving a selection of a facility, the GUI 600 may be updated to display the actual and/or predicted facility score(s) at one or more points in time, the salient feature(s) at one or more points in time, and the like. In some embodiments, the GUI 600 can be similarly used to trigger the generation of a new predicted score (e.g., based on new data), as discussed above with reference to FIG. 2. In some embodiments, the GUI 600 can be similarly used to experiment or test out different values for various features in order to predict their effect on the score, as discussed above with reference to FIG. 3.

Generally, the GUI 600 may be used to enable interactivity with any aspects of the present disclosure, providing rapid, dynamic, and efficient evaluation and review of the various data and predictions in order to enable more efficient and timely updates and configuration changes.

### Example Method for Generating Predictive Models for Operational Scoring

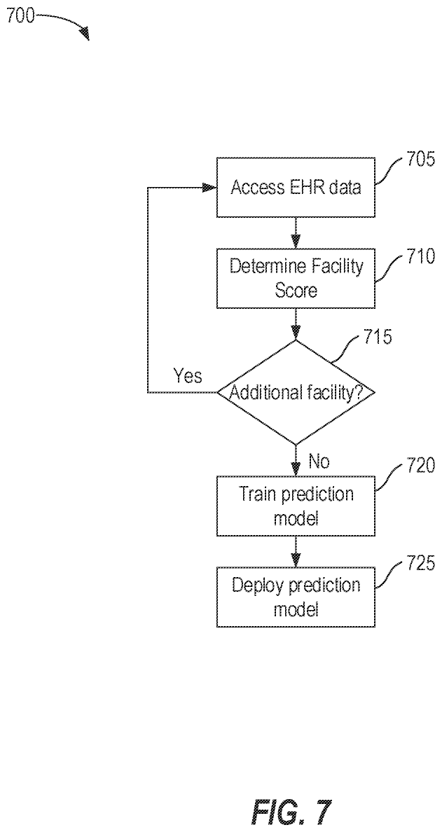

FIG. 7 is a flow diagram depicting an example method 700 for generating predictive models for operational scoring, according to some embodiments. In some embodiments, the method 700 is performed by a scoring system, such as scoring system 120 of FIG. 1 and/or the scoring system 220 of FIGS. 2-3. In one embodiment, the method 700 provides additional detail for the workflow 100 of FIG. 1.

At block 705, the scoring system accesses EHR data from one or more facilities. For example, as discussed above, the scoring system may retrieve, receive, request, collect, or otherwise access data (e.g., EHR data 110 of FIG. 1) directly or indirectly from one or more healthcare facilities (e.g., healthcare facilities 105 of FIG. 1). In some embodiments, as discussed above, the facilities correspond to long-term residential care facilities. In an embodiment, the EHR data comprises or corresponds to various records relating to health information for various patients or residents of such facilities. As discussed above, the particular contents of the EHR data may vary depending on the particular implementation. In some embodiments, the EHR data may include, for one or more patients or residents, information such as their demographics, medications, test results, allergies, medical history, vital signs collected or recorded at one or more times, progress notes recorded (e.g., by a care provider) at one or more times, and the like.

In some embodiments, as discussed above, the EHR data may include (or may be used to generate) aggregated feature data for the healthcare facility in addition to or instead of including patient-specific data. For example, the scoring system may determine aggregated attributes such as the average number of progress notes recorded per resident, the average demographics, the aggregated medication usage of the residents and/or frequency of medication administration issues in the facility, and the like. Such aggregated features can then be used as model input.

At block 710, the scoring system determines the facility score (e.g., facility score 115 of FIG. 1) for the facility that corresponds to the accessed EHR data. As discussed above, the facility score generally comprises or corresponds to one or more measures that quantify the healthcare facility based on its operations or quality. For example, the facility score may quantify the quality, efficiency, efficacy, or other aspects of the facility. In some embodiments, as discussed above, the facility score may be generated or created based on surveys or other data from residents, patients, and/or staff at the healthcare facility.

At block 715, the scoring system determines whether there is at least one additional facility, for which EHR data is available for training, that has not-yet been accessed. That is, the scoring system determines whether there is training EHR data available from one more additional facilities. If not, the method 700 continues to block 720. If so, the method 700 returns to block 715 to access EHR data from the facility. In an embodiment, if multiple such facilities exist, the scoring system may select the facility using any suitable technique, including randomly or pseudo-randomly, as all available training data may be accessed during the method 700. Further, though the illustrated example depicts a sequential process for conceptual clarity (e.g., where data from each facility is accessed in turn), in some embodiments, the scoring system may access some or all of the facility data in parallel.

At block 720, the scoring system generates and/or trains a prediction model based on the accessed EHR data for one or more facilities and the determined facility scores for the one or more facilities. For example, in some embodiments, the scoring system uses the facility score as the target output of the model, while using some or all of the EHR data as input. In some embodiments, as discussed above, the scoring system evaluates the accessed EHR data to generate or extract a set of features. Based on the input features, a predicted score is generated, and this predicted score can be compared against the determined facility score to generate a loss used to refine the parameters of the model.

In some embodiments, as discussed above, the scoring system can use a subset of the EHR data as input, or may evaluate or parse the EHR data to extract or generate features used as input. In some embodiments, the scoring system (or another system) may generate facility-wide features by aggregating information found in the EHR data for multiple residents. For example, as discussed above, the input features may include facility features such as the total number of residents, the number of progress notes recorded per resident (or the frequency of such notes), the frequency with which vitals are collected per resident, the frequency with which medication administration issues arise, and the like. Such aggregated features can then be used as model input.

In at least one embodiment, training the prediction model can include identifying and using a subset of the features to train the model. For example, the scoring system may use one or more feature selection operations or techniques to identify or select a subset of salient or useful features, and use this subset of features to train the model (where other features or aspects of the EHR data may be ignored or discarded during training).

In this way, as discussed above, the scoring system can additionally or alternatively generate or train static models, dynamic models, and the like in order to effectively generate predicted quality scores for facilities based on the aggregated EHR data.

At block 725, once training is complete, the scoring system deploys the prediction model for runtime inferencing. As discussed above, deploying the model generally includes providing and/or instantiating it for runtime, either locally (e.g., for inferencing by the scoring system) or remotely (e.g., for inferencing by one or more other systems). Once deployed, the prediction model can be used to generate predicted scores based on input features (e.g., input EHR data), as discussed above.

### Example Method for Generating Predicted Scores Using Predictive Models

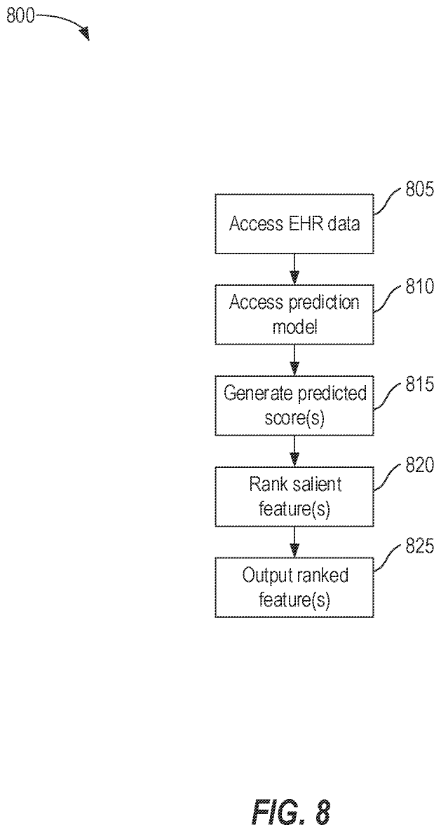

FIG. 8 is a flow diagram depicting an example method 800 for generating predicted scores using predictive models, according to some embodiments. In some embodiments, the method 800 is performed by a scoring system, such as scoring system 120 of FIG. 1 and/or the scoring system 220 of FIGS. 2-3. In one embodiment, the method 800 provides additional detail for the workflow 200 of FIG. 1.

At block 805, the scoring system accesses EHR data (e.g., EHR data 210 of FIG. 2) from a facility. In some embodiments, as discussed above, the facility corresponds to a long-term residential care facility (e.g., a nursing home or senior living center) or other healthcare facility. In some embodiments, as discussed above, the EHR data can generally comprise or correspond to various records relating to health information for various patients or residents. For example, the EHR data may include, for one or more patients or residents, information such as their demographics, medications, test results, allergies, medical history, vital signs collected or recorded at one or more times, progress notes recorded (e.g., by a care provider) at one or more times, and the like. In some embodiments, in addition to or instead of including patient-specific data, the EHR data may include (or may be used to generate) aggregated data for the healthcare facility, as discussed above.

At block 810, the scoring system accesses a prediction model to generate a predicted facility score. For example, as discussed above, the scoring system may use a locally deployed model that was trained or generated by the scoring system itself, or by another system. In some embodiments, accessing the prediction model includes using a remote model. For example, the prediction model may be hosted or provided on another system (e.g., in the cloud), and the scoring system may transmit some or all of the EHR data (e.g., aggregated features generated based on the EHR data) to the remote system to generate the prediction.

At block 815, the scoring system generates one or more predicted scores (e.g., predicted score 225 of FIG. 2) for the facility based on processing the EHR data (or generated features therefrom) using the prediction model. As discussed above, the generated predicted score(s) are generally indicative of the predicted, probable, or likely quality of the facility. In some embodiments, the scoring system generates a single score (e.g., by processing the EHR features). In at least one embodiment, the scoring system can generate multiple scores (e.g., by perturbing the features and generating a separate score for each variation).

At block 820, the scoring system can rank the some or all of the features, used by the model and reflected by the EHR data, based on their contribution or impact to the predicted scores. In some embodiments, the scoring system ranks all such features. In some embodiments, the scoring system can identify and rank the salient or most-impactful features (discarding or ignoring the least impactful). As discussed above, the scoring system may use a variety of techniques to determine and/or rank the features based on their salience or contribution.

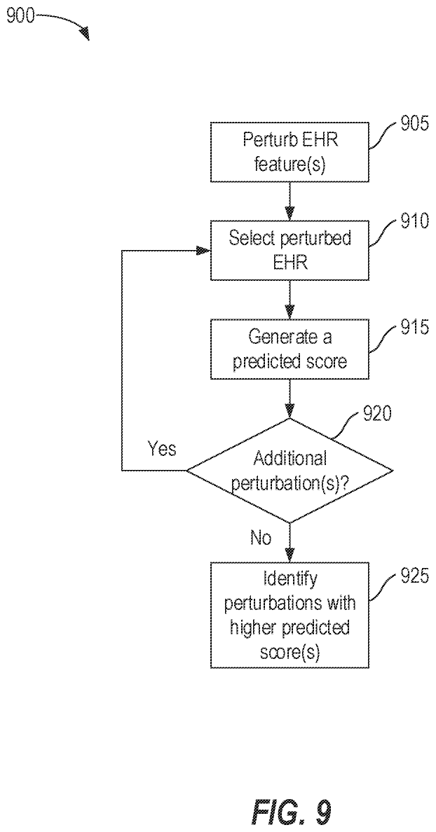

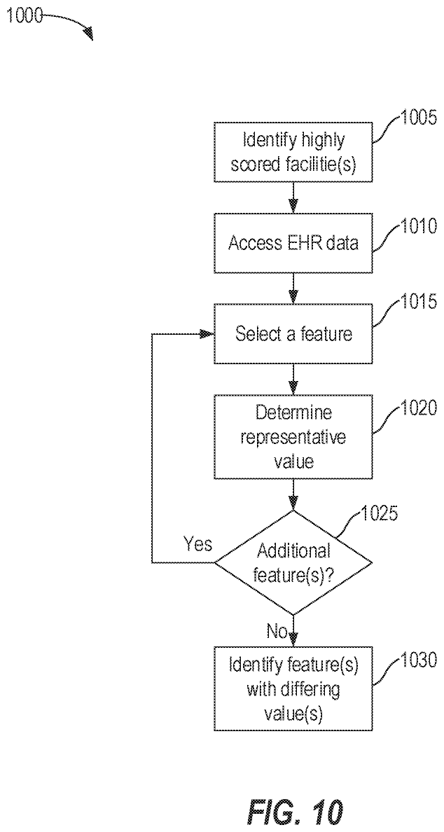

For example, in some embodiments, the scoring system (or another system) may use a variety of feature selection operations or techniques (e.g., during training) to identify the contribution or importance of each EHR feature. In some embodiments, as discussed above and below with reference to FIG. 9, the scoring system may rank the features by generating multiple predicted scores using perturbed or modified EHR data, identifying impactful features for the specific facility based on the resulting scores. In some embodiments, as discussed above and below with reference to FIG. 10, the scoring system may rank the features by comparing the features of the current facility (e.g., the EHR data accessed at block 805) to a set of features that are representative of high quality facilities, identifying the feature(s) that differ above a threshold from the representative values.

At block 825, the scoring system outputs the predicted score and/or ranked feature(s). For example, as discussed above, the scoring system may output the features via a GUI (e.g., GUI 600 of FIG. 6), allowing a user or administrator to efficiently evaluate not only the (predicted) quality of the facility based on the current data, but also which aspects of the facility could be changed or reconfigured to improve the quality.

In some embodiments, the scoring system generates and/or outputs the ranked features based on determining that the predicted score and/or the actual score of the facility satisfies one or more criteria. For example, the scoring system may output the ranked features in response to determining that the predicted score and/or actual score fall below a threshold (e.g., indicating that changes may be useful, and that outputting the ranked features may be beneficial). In some embodiments, the criteria relates to whether the predicted score exists/was successfully generated (e.g., the input was not missing data or malformed), or whether a confidence value associated with the prediction satisfies defined criteria. In some embodiments, the criteria relate to whether a user has indicated that they would like more information on how to improve the actual score and/or predicted score. Generally, the criteria may include a wide variety of considerations in various embodiments.

### Example Method for Ranking Impactful Features Using Local Evaluations

FIG. 9 is a flow diagram depicting an example method 900 for ranking impactful features using local evaluations, according to some embodiments. In some embodiments, the method 900 is performed by a scoring system, such as scoring system 120 of FIG. 1 and/or the scoring system 220 of FIGS. 2-3. In one embodiment, the method 900 provides additional detail for the workflow 400 of FIG. 4 and/or for blocks 815 and 820 of FIG. 8.

At block 905, the scoring system perturbs one or more features of the current EHR data for the facility. As discussed above, perturbing the EHR features can generally include modifying one or more values of the features (e.g., the value of one or more dimensions in the features). For example, the scoring system may increase the reported frequency of check-ins, decrease the staff-to-resident ratio, and the like. In some embodiments, the scoring system perturbs only a single feature for each set of perturbed features. In other embodiments, the scoring system may perturb multiple features within each set of perturbed features.

In some embodiments, as discussed above, the scoring system perturbs the features using a random or pseudo-random process. For example, the scoring system may use a random process to select the number of features to be perturbed, use a random process to select the specific feature(s) to be perturbed, use a random process to determine the amount of perturbation for each feature, and the like. In at least one embodiment, the scoring system perturbs the features based at least in part on the distribution of values seen, for each feature, during training. In this way, the scoring system can ensure that the perturbed features are “realistic” in that it is feasible for them to be reflected in a real facility.

At block 910, the scoring system selects a set of perturbed EHR features. Generally, the scoring system can use any suitable technique to select the perturbed features, as all sets of perturbed features will be evaluated using the method 900. Although the illustrated example depicts an iterative process (selecting and evaluating each set of perturbed features in turn) for conceptual clarity, in some embodiments, the scoring system may select and process multiple features in parallel.

At block 915, the scoring system generates a predicted score for the selected set of perturbed features using the prediction model. The method 900 then continues to block 920, where the scoring system determines whether there is at least one more set of perturbations that have not-yet been evaluated. If so, the method 900 returns to block 910. If not, the method 900 continues to block 925.

At block 925, the scoring system identifies perturbations (e.g., sets of perturbed features) associated with predicted scores that are higher than the predicted score generated using the actual (unperturbed) EHR data. The scoring system can then identify the specific changes/perturbations reflected in these identified sets. In some embodiments, the specific features that were perturbed can be provided as the salient or impactful features, in that the predicted score increased when the specific features were changed.

In this way, the scoring system can identify specific changes and specific salient features based on the specific EHR data for the current facility. That is, the salient features may differ depending on the particular facility at the particular time. In this way, the same facility may have different salient features at a different time (when different EHR data is used), and a different facility may have different salient features at the same time (because it has different EHR data).

This can enable highly dynamic and targeted configurations and updates, as the determined impactful features are specifically determined for the facility at runtime.

### Example Method for Ranking Impactful Features Using Global Evaluations

FIG. 10 is a flow diagram depicting an example method 1000 for ranking impactful features using global evaluations, according to some embodiments. In some embodiments, the method 1000 is performed by a scoring system, such as scoring system 120 of FIG. 1 and/or the scoring system 220 of FIGS. 2-3. In one embodiment, the method 1000 provides additional detail for the workflow 500 of FIG. 5 and/or for blocks 815 and 820 of FIG. 8.

At block 1005, the scoring system identifies a set of highly scored facilities. For example, as discussed above, the scoring system may identify facilities having actual or predicted quality scores that satisfy one or more criteria, such as meeting or exceeding a threshold quality.

At block 1010, the scoring system accesses EHR data for the identified facilities. For example, as discussed above, the scoring system may retrieve, collect, request, or otherwise gain access to the EHR data associated with the set of facilities that are determined to be high quality.

At block 1015, the scoring system selects one of the EHR features used by the prediction model. In some embodiments, the scoring system may use any suitable technique to select the feature, including randomly or pseudo-randomly, as all of the features will be evaluated during the method 1000. Although the illustrated example depicts an iterative process (selecting and evaluating each feature in turn) for conceptual clarity, in some embodiments, the scoring system may process some or all of the features in parallel.

At block 1020, the scoring system determines a representative or aggregate value for the selected feature based on the EHR data from the highly scored facilities. For example, as discussed above, the scoring system may determine the average or mean value, the median value, the variance or distribution of values, and the like.

At block 1025, the scoring system determines whether there is at least one additional feature remaining to be evaluated. If so, the method 1000 returns to block 1015. If not, the method 1000 continues to block 1030, where the scoring system identifies any features, reflected in the EHR data for the current facility, having values that differ from the determined representative values of high-scoring facilities.

In some embodiments, the scoring system determines, for each feature, the difference between the representative value and the actual value for the facility. In at least one embodiment, the scoring system can compare these differences against one or more thresholds (which may include fixed thresholds, variable thresholds based on the actual value and/or distribution of values, and the like). If the difference for a given feature satisfies the criteria, the scoring system can determine that the given feature is relevant or salient, in that the value of the feature, with respect to the specific facility, differs substantially from the representative value of high-scoring facilities.

As discussed above, these features with differing values can then be provided as the set of salient, relevant, or impactful features for the facility. That is, because the features differ from the representative values of high-scoring facilities, the scoring system can infer or determine that they may be at least partially responsible for the current score of the facility. Therefore, such features may be a desirable target for modification or reconfiguration to improve the score of the facility.

### Example Method for Generating Updated Predictions for Newly Received Data

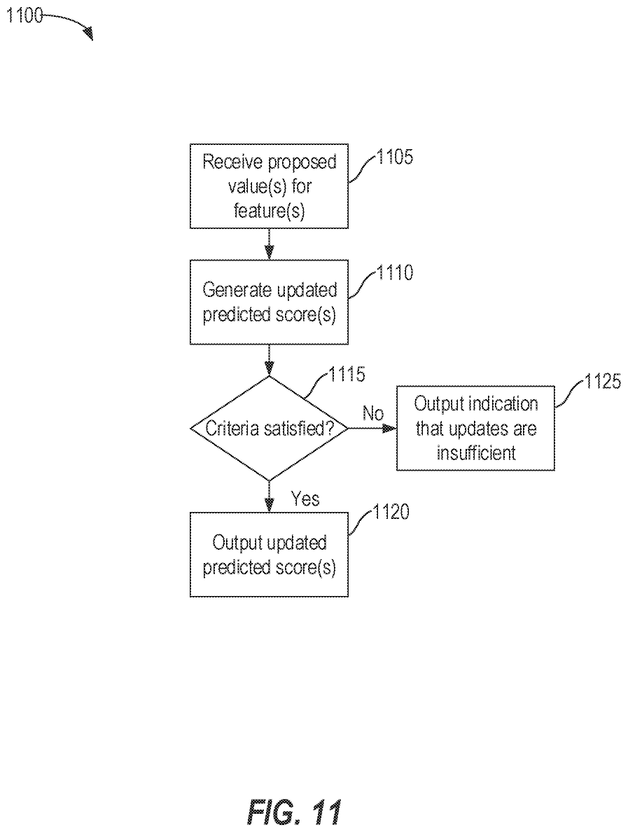

FIG. 11 is a flow diagram depicting an example method 1100 for generating updated predictions for newly received data, according to some embodiments. In some embodiments, the method 1100 is performed by a scoring system, such as scoring system 120 of FIG. 1 and/or the scoring system 220 of FIGS. 2-3. In one embodiment, the method 1100 provides additional detail for the workflow 300 of FIG. 3.

At block 1105, the scoring system receives a set of proposed values for one or more features for a facility. For example, as discussed above, before or after generating a predicted score based on the facility's current EHR data, the scoring system may receive proposed or potential values (e.g., from a user) for one or more features. In at least one embodiment, these proposed or potential values are provided based at least in part on the determined salient or impactful features for the facility. In some embodiments, the scoring system receives the proposed values from a user. In at least one embodiment, the scoring system itself generates the proposed values. For example, in response to determining that a given feature is impactful for the facility, the scoring system may generate a proposed new value for the feature.

At block 1110, the scoring system generates one or more updated predicted scores based on the proposed value(s) and the remaining EHR data for the facility. For example, as discussed above, the scoring system may use a prediction model to generate the predicted score.

At block 1115, the scoring system determines whether one or more criteria are satisfied. In one embodiment, the criteria relate to whether the updated predicted score is higher than the current predicted score (using current/unmodified features for the facility), or whether the difference between the updated score and the current score exceeds some magnitude. If not, the method 1100 continues to block 1125, where the scoring system outputs an indication (e.g., via the GUI 600 of FIG. 6) that the proposed updated feature values are insufficient to improve the score (or to improve it sufficiently).

If, at block 1115, the scoring system determines that the criteria are satisfied, the method 1100 continues to block 1120, where the scoring system outputs the updated predicted score(s) (e.g., via the GUI 600 of FIG. 6). In this way, the user can readily determine which potential changes should be made in order to improve the quality of the facility.

### Example Method for Refining Predictive Models Over Time

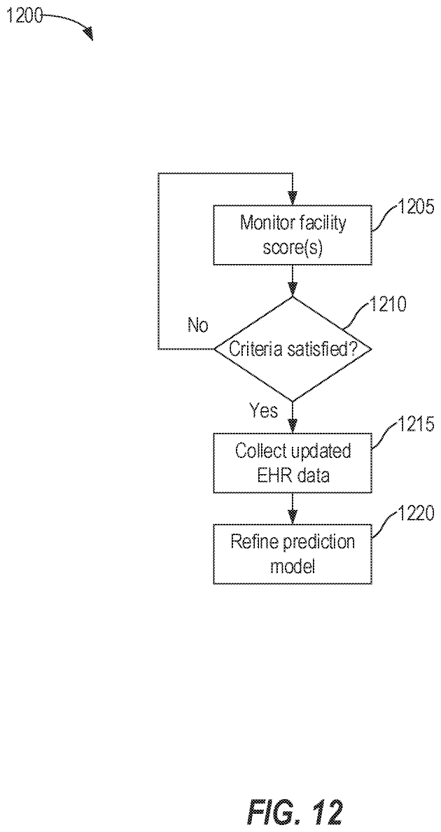

FIG. 12 is a flow diagram depicting an example method 1200 for refining predictive models over time, according to some embodiments. In some embodiments, the method 1200 is performed by a scoring system, such as scoring system 120 of FIG. 1 and/or the scoring system 220 of FIGS. 2-3.

At block 1205, the scoring system monitors facility scores for one or more facilities. For example, as discussed above, the scoring system (or another system) may periodically generate ratings or scores for the facilities based on survey responses or other data. In some embodiments, the scoring system monitors these generated scores over time, such as to determine whether new scores are available, whether the new scores differ from the old ones, and the like.

At block 1210, the scoring system determines whether one or more criteria are satisfied by the monitoring. For example, as discussed above, the scoring system may determine whether new scores have been created. In some embodiments, the scoring system determines whether the newly created scores differ from the prior scores, or differ beyond a threshold amount.

If, at block 1210, the scoring system determines that the criteria are not satisfied, the method 1200 returns to block 1205. If the scoring system determines that the criteria are satisfied, the method 1200 continues to block 1215, where the scoring system collects or accesses updated EHR data for the relevant facility (or facilities). That is, for each facility having a new score (or a new score that satisfies the criteria), the scoring system can access corresponding EHR data. In some embodiments, the scoring system collects the current EHR data of the facility. In at least one embodiment, the scoring system can identify/access EHR data that was relevant as of the time when the score was generated. For example, if the score was generated based on survey responses received during a defined window of time, the scoring system may access the EHR data from that window of time.

At block 1220, the scoring system can then refine the prediction model based on the updated EHR data and new score(s). For example, as discussed above, the scoring system may update or refine one or more parameters of the model based on the updated scores and data. In at least one embodiment, the scoring system does so by using the new scores and data as new training exemplars, as discussed above.

In this way, the scoring system can continue to update the model as new data becomes available, ensuring it remains relevant and accurate as conditions change.

### Example Method for Scoring and Ranking Features Using Predictive Models

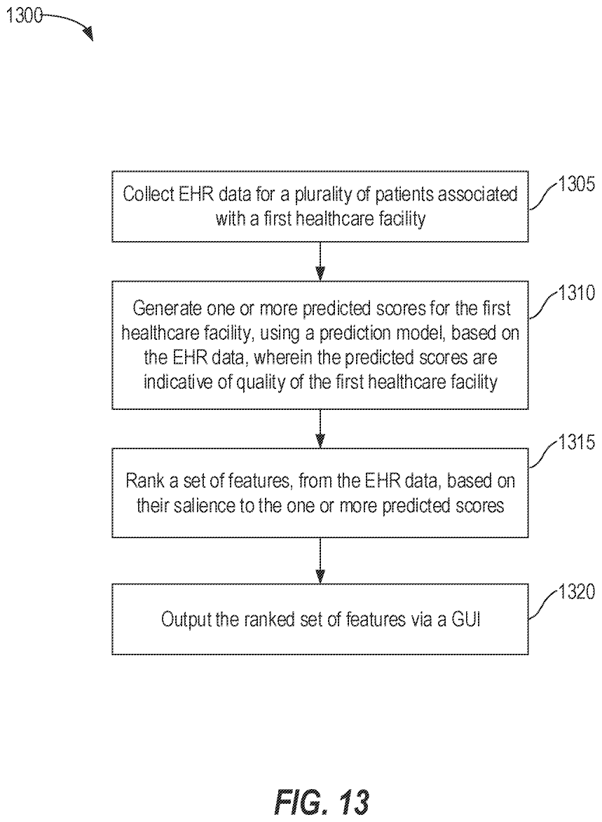

FIG. 13 is a flow diagram depicting an example method 1300 for scoring and ranking features using predictive models, according to some embodiments. In some embodiments, the method 1300 is performed by a scoring system, such as scoring system 120 of FIG. 1 and/or the scoring system 220 of FIGS. 2-3.

At block 1305, EHR data (e.g., EHR data 210 of FIG. 2) for a plurality of patients associated with a first healthcare facility (e.g., healthcare facility 205 of FIG. 2) is collected.

At block 1310, one or more predicted scores (e.g., predicted score 225 of FIG. 2) for the first healthcare facility are generated using a prediction model (e.g., prediction model 135 of FIG. 1), based on the EHR data, wherein the predicted scores are indicative of quality of the first healthcare facility.

At block 1315, a set of features (e.g., features 230 of FIG. 2), from the EHR data, is ranked based on their salience to the one or more predicted scores.

At block 1320, the ranked set of features is output via a GUI (e.g., GUI 600 of FIG. 6).

### Example Method for Training Predictive Models to Predict Operational Scores

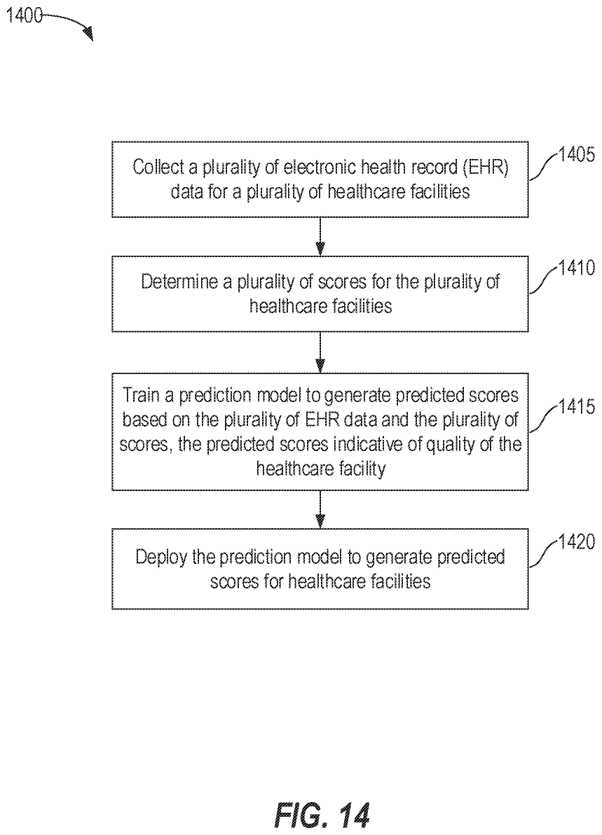

FIG. 14 is a flow diagram depicting an example method 1400 for training predictive models to predict operational scores, according to some embodiments. In some embodiments, the method 1400 is performed by a scoring system, such as scoring system 120 of FIG. 1 and/or the scoring system 220 of FIGS. 2-3.

At block 1405, a plurality of EHR data (e.g., EHR data 110 of FIG. 1) for a plurality of healthcare facilities (e.g., healthcare facilities 105 of FIG. 1) is collected.

At block 1410, a plurality of scores (e.g., facility scores 115 of FIG. 1) is determined for the plurality of healthcare facilities.

At block 1415, a prediction model (e.g., prediction model 135 of FIG. 1) is trained to generate predicted scores based on the plurality of EHR data and the plurality of scores, the predicted scores indicative of quality of the healthcare facility.

At block 1420, the prediction model is deployed to generate predicted scores for healthcare facilities.

### Example Processing System for Improved Predictive Modeling

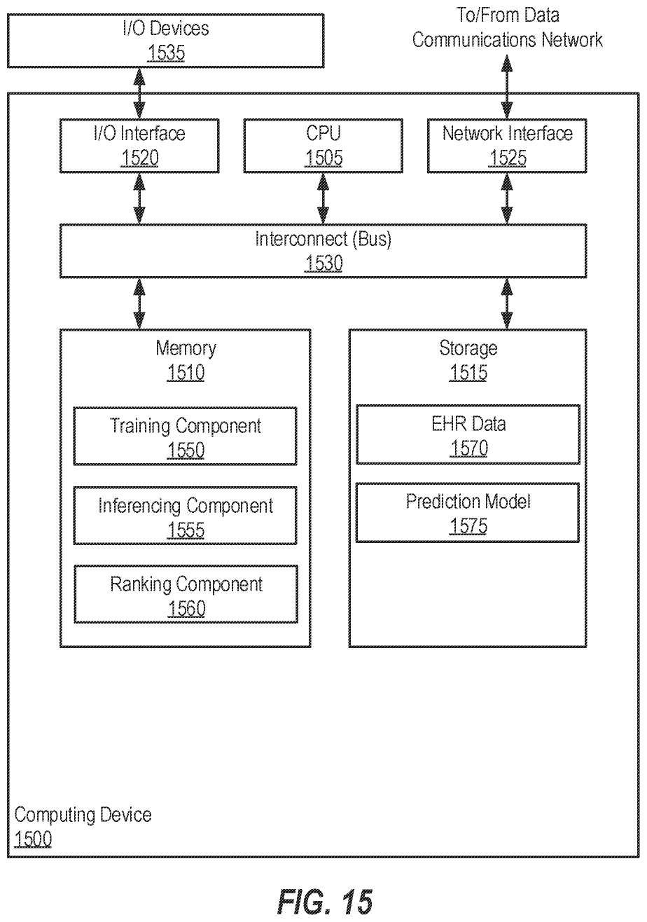

FIG. 15 depicts an example computing device 1500 configured to perform various aspects of the present disclosure, according to some embodiments. Although depicted as a physical device, in embodiments, the computing device 1500 may be implemented using virtual device(s), and/or across a number of devices (e.g., in a cloud environment). In one embodiment, the computing device 1500 corresponds to the scoring system 120 of FIG. 1 and/or the scoring system 220 of FIGS. 2-3.

As illustrated, the computing device 1500 includes a CPU 1505, memory 1510, storage 1515, a network interface 1525, and one or more input/output (I/O) interfaces 1520. In the illustrated embodiment, the CPU 1505 retrieves and executes programming instructions stored in memory 1510, as well as stores and retrieves application data residing in storage 1515. The CPU 1505 is generally representative of a single CPU and/or GPU, multiple CPUs and/or GPUs, a single CPU and/or GPU having multiple processing cores, and the like. The memory 1510 is generally included to be representative of a random access memory. Storage 1515 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 1535 (such as keyboards, monitors, etc.) are connected via the I/O interface(s) 1520. Further, via the network interface 1525, the computing device 1500 can be communicatively coupled with one or more other devices and components (e.g., via a network, which may include the Internet, local network(s), and the like). As illustrated, the CPU 1505, memory 1510, storage 1515, network interface(s) 1525, and I/O interface(s) 1520 are communicatively coupled by one or more buses 1530.

In the illustrated embodiment, the memory 1510 includes a training component 1550, an inferencing component 1555, and a ranking component 1560, which may perform one or more embodiments discussed above. Although depicted as discrete components for conceptual clarity, in embodiments, the operations of the depicted components (and others not illustrated) may be combined or distributed across any number of components. Further, although depicted as software residing in memory 1510, in embodiments, the operations of the depicted components (and others not illustrated) may be implemented using hardware, software, or a combination of hardware and software.

In one embodiment, the training component 1550 is used to generate training data (e.g., to perform feature extraction on EHR data) and/or to train or generate computational models (e.g., prediction models), as discussed above. The inferencing component 1555 may generally be used to generate predicted quality scores for facilities based on EHR data (or features extracted therefrom), as discussed above. The ranking component 1560 may be configured to determine the contribution or impact of the features, with respect to a given facility, as discussed above.

In the illustrated example, the storage 1515 includes EHR data 1570 (which may correspond to EHR data 110 of FIG. 1, EHR data 210 of FIG. 2, EHR data 310 of FIG. 3, EHR data 410 of FIG. 4. EHR data 510 of FIG. 5, and the like). The storage 1515 also includes one or more prediction models 1575 (which may correspond to the prediction model 135 of FIG. 1). Although depicted as residing in storage 1515, the EHR data 1570 and prediction model 1575 may be stored in any suitable location, including memory 1510.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access applications or systems (e.g., the scoring system 120 and/or the scoring system 220) or related data available in the cloud. For example, the scoring system could execute on a computing system in the cloud and generate and/or use predictive models. In such a case, the scoring system could train models to generate operational scores, and store the models at a storage location in the cloud. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

### Example Clauses

Implementation examples are described in the following numbered clauses:

Clause 1: A method, comprising: collecting electronic health record (EHR) data for a plurality of patients associated with a first healthcare facility; generating one or more predicted scores for the first healthcare facility, using a prediction model, based on the EHR data, wherein the predicted scores are indicative of quality of the first healthcare facility; ranking a set of features, from the EHR data, based on their salience to the one or more predicted scores; and outputting the ranked set of features via a graphical user interface (GUI).

Clause 2: The method of Clause 1, wherein generating one or more predicted scores for the first healthcare facility comprises: generating a plurality of perturbed EHR data by modifying one or more features of the EHR data; and generating a plurality of predicted scores by processing the plurality of perturbed EHR data using the prediction model, wherein ranking the set of features comprises determining, for each respective feature in the set of features, a respective contribution to one or more predicted scores in the plurality of predicted scores.

Clause 3: The method of any one of Clauses 1-2, wherein the prediction model comprises a trained machine learning model that was trained based on EHR data for a plurality of healthcare facilities.

Clause 4: The method of any one of Clauses 1-3, further comprising: identifying a subset of high-performing healthcare facilities, from a plurality of healthcare facilities, based on comparing a plurality of scores to one or more criteria; and ranking the set of features based at least in part on EHR data from healthcare facilities in the subset of high-performing healthcare facilities.

Clause 5: The method of any one of Clauses 1-4, wherein ranking the set of features comprises, for each respective feature in the set of features: determining a respective representative value with respect to the EHR data from the subset of high-performing healthcare facilities; and determining a respective difference between the respective representative value and a value of the respective feature for the first healthcare facility.

Clause 6: The method of any one of Clauses 1-5, further comprising: receiving, via the GUI, an updated value for one or more features of the set of features; generating an updated predicted score for the first healthcare facility, using the prediction model, based on the updated value; and outputting the updated predicted score via the GUI.

Clause 7: The method of any one of Clauses 1-6, wherein the set of features comprise at least one of: a number of notes recorded for each patient, of the plurality of patients, during a duration of time; a number of times one or more vitals are recorded for each patient, of the plurality of patients, during the duration of time; a frequency of medication administration issues; or a frequency of falls experienced by one or more patients of the plurality of patients.

Clause 8: The method of any one of Clauses 1-7, wherein: the first healthcare facility corresponds to a residential care facility, and the plurality of patients correspond to residents of the residential care facility.

Clause 9: A method, comprising: collecting a plurality of electronic health record (EHR) data for a plurality of healthcare facilities; determining a plurality of scores for the plurality of healthcare facilities; training a prediction model to generate predicted scores based on the plurality of EHR data and the plurality of scores, the predicted scores indicative of quality of the healthcare facility; and deploying the prediction model to generate predicted scores for healthcare facilities.

Clause 10: The method of Clause 9, further comprising: identifying a set of features in the plurality of EHR data; selecting a subset of salient features, from the set of features, using one or more feature selection operations; and training the prediction model based on the subset of salient features.

Clause 11: The method of any one of Clauses 9-10, wherein the plurality of EHR data comprise, for each respective healthcare facility of the plurality of healthcare facilities, at least one of: a respective number of notes recorded for each patient of the respective healthcare facility during a duration of time; a respective number of times one or more vitals were recorded for each patient of the respective healthcare facility during the duration of time; a frequency of medication administration issues; or a respective frequency of falls experienced by one or more patients of the plurality of patients.

Clause 12: The method of any one of Clauses 9-11, wherein the plurality of healthcare facilities correspond to residential care facilities.

Clause 13: A system, comprising: a memory comprising computer-executable instructions; and one or more processors configured to execute the computer-executable instructions and cause the processing system to perform a method in accordance with any one of Clauses 1-12.

Clause 14: A system, comprising means for performing a method in accordance with any one of Clauses 1-12.

Clause 15: A non-transitory computer-readable medium comprising computer-executable instructions that, when executed by one or more processors of a processing system, cause the processing system to perform a method in accordance with any one of Clauses 1-12.

Clause 16: A computer program product embodied on a computer-readable storage medium comprising code for performing a method in accordance with any one of Clauses 1-12.

What is claimed is:

1. A method, comprising:
collecting electronic health record (EHR) data for a plurality of patients associated with a first healthcare facility;
generating one or more predicted scores for the first healthcare facility, using a prediction model, based on the EHR data, wherein the predicted scores are indicative of quality of the first healthcare facility;
ranking a set of features, from the EHR data, based on their salience to the one or more predicted scores; and
outputting the ranked set of features via a graphical user interface (GUI).

2. The method of claim 1, wherein generating one or more predicted scores for the first healthcare facility comprises:
generating a plurality of perturbed EHR data by modifying one or more features of the EHR data; and
generating a plurality of predicted scores by processing the plurality of perturbed EHR data using the prediction model, wherein ranking the set of features comprises determining, for each respective feature in the set of features, a respective contribution to one or more predicted scores in the plurality of predicted scores.

3. The method of claim 1, wherein the prediction model comprises a trained machine learning model that was trained based on EHR data for a plurality of healthcare facilities.

4. The method of claim 1, further comprising:
identifying a subset of high-performing healthcare facilities, from a plurality of healthcare facilities, based on comparing a plurality of scores to one or more criteria; and
ranking the set of features based at least in part on EHR data from healthcare facilities in the subset of high-performing healthcare facilities.

5. The method of claim 4, wherein ranking the set of features comprises, for each respective feature in the set of features:
determining a respective representative value with respect to the EHR data from the subset of high-performing healthcare facilities; and
determining a respective difference between the respective representative value and a value of the respective feature for the first healthcare facility.

6. The method of claim 1, further comprising:
receiving, via the GUI, an updated value for one or more features of the set of features;
generating an updated predicted score for the first healthcare facility, using the prediction model, based on the updated value; and
outputting the updated predicted score via the GUI.

7. The method of claim 1, wherein the set of features comprise at least one of:
a number of notes recorded for each patient, of the plurality of patients, during a duration of time;
a number of times one or more vitals are recorded for each patient, of the plurality of patients, during the duration of time;
a frequency of medication administration issues; or
a frequency of falls experienced by one or more patients of the plurality of patients.

8. The method of claim 1, wherein:
the first healthcare facility corresponds to a residential care facility, and
the plurality of patients correspond to residents of the residential care facility.

9. A method, comprising:
collecting a plurality of electronic health record (EHR) data for a plurality of healthcare facilities;
determining a plurality of scores for the plurality of healthcare facilities;
training a prediction model to generate predicted scores based on the plurality of EHR data and the plurality of scores, the predicted scores indicative of quality of the healthcare facility; and
deploying the prediction model to generate predicted scores for healthcare facilities.

10. The method of claim 9, further comprising:
identifying a set of features in the plurality of EHR data;
selecting a subset of salient features, from the set of features, using one or more feature selection operations; and
training the prediction model based on the subset of salient features.

11. The method of claim 9, wherein the plurality of EHR data comprise, for each respective healthcare facility of the plurality of healthcare facilities, at least one of:
a respective number of notes recorded for each patient of the respective healthcare facility during a duration of time;
a respective number of times one or more vitals were recorded for each patient of the respective healthcare facility during the duration of time;
a respective frequency of medication administration issues; or
a respective frequency of falls experienced by one or more patients of the plurality of patients.

12. The method of claim 9, wherein the plurality of healthcare facilities correspond to residential care facilities.

13. A non-transitory computer-readable storage medium comprising computer-readable program code that, when executed using one or more computer processors, performs an operation comprising:
collecting electronic health record (EHR) data for a plurality of patients associated with a first healthcare facility;
generating one or more predicted scores for the first healthcare facility, using a prediction model, based on the EHR data, wherein the predicted scores are indicative of quality of the first healthcare facility;
ranking a set of features, from the EHR data, based on their salience to the one or more predicted scores; and
outputting the ranked set of features via a graphical user interface (GUI).

14. The non-transitory computer-readable storage medium of claim 13, wherein generating one or more predicted scores for the first healthcare facility comprises:
generating a plurality of perturbed EHR data by modifying one or more features of the EHR data; and
generating a plurality of predicted scores by processing the plurality of perturbed EHR data using the prediction model, wherein ranking the set of features comprises determining, for each respective feature in the set of features, a respective contribution to one or more predicted scores in the plurality of predicted scores.

15. The non-transitory computer-readable storage medium of claim 13, wherein the prediction model comprises a trained machine learning model that was trained based on EHR data for a plurality of healthcare facilities.

16. The non-transitory computer-readable storage medium of claim 13, the operation further comprising:
identifying a subset of high-performing healthcare facilities, from a plurality of healthcare facilities, based on comparing a plurality of scores to one or more criteria; and
ranking the set of features based at least in part on EHR data from healthcare facilities in the subset of high-performing healthcare facilities.

17. The non-transitory computer-readable storage medium of claim 16, wherein ranking the set of features comprises, for each respective feature in the set of features:
determining a respective representative value with respect to the EHR data from the subset of high-performing healthcare facilities; and
determining a respective difference between the respective representative value and a value of the respective feature for the first healthcare facility.

18. The non-transitory computer-readable storage medium of claim 13, the operation further comprising:
receiving, via the GUI, an updated value for one or more features of the set of features;
generating an updated predicted score for the first healthcare facility, using the prediction model, based on the updated value; and
outputting the updated predicted score via the GUI.

19. The non-transitory computer-readable storage medium of claim 13, wherein the set of features comprise at least one of:
a number of notes recorded for each patient, of the plurality of patients, during a duration of time;
a number of times one or more vitals are recorded for each patient, of the plurality of patients, during the duration of time;
a frequency of medication administration issues; or
a frequency of falls experienced by one or more patients of the plurality of patients.

20. The non-transitory computer-readable storage medium of claim 13, wherein:
the first healthcare facility corresponds to a residential care facility, and
the plurality of patients correspond to residents of the residential care facility.
